(19) United States
(12) Patent Application Publication
(10) Pub. No.: US 2021/0196984 A1
(43) Pub. Date: Jul. 1, 2021

(54) ACCELERATOR AND PARTICLE THERAPY SYSTEM INCLUDING THEREOF

(71) Applicant: HITACHI, LTD., Tokyo (JP)

(72) Inventors: Takamitsu HAE, Tokyo (JP); Takamichi AOKI, Tokyo (JP); Takayoshi SEKI, Tokyo (JP)

(21) Appl. No.: 17/057,161

(22) PCT Filed: Feb. 18, 2019
(86) PCT No.: PCT/JP2019/005849

§ 371 Date: Nov. 20, 2020

(30) Foreign Application Priority Data
Sep. 4, 2018 (JP) 2018-165482

(51) Int. Cl. A61N 5/10; H05H 7/10; H05H 13/00; H05H 7/04; H05H 13/02

(52) U.S. Cl. CPC A61N 5/1078; H05H 7/10; H05H 13/02; H05H 7/04; H05H 13/005

(57) ABSTRACT

An acceleration radiofrequency acceleration system capable of frequency modulation and feeding an acceleration radiofrequency wave for accelerating a beam, a radiofrequency kicker 70 feeding an extraction radiofrequency wave different in frequency, from the acceleration radiofrequency wave for extracting a beam, a peeler magnetic field region 44 and a regenerator magnetic field region 45 for forming a disturbance magnetic field region including a high-order magnetic field that includes a magnetic field component having a number of poles of two poles or more and that includes at least a quadrupole magnetic field component, a shim of a magnetic material, and a septum magnet 43, 43A, 43B having an inner peripheral side septum coil conductor 5, an outer peripheral side septum coil conductor 6, a coil conductor connecting portion 7, and a coil lead-out portion 8 are provided.

### TECHNICAL FIELD

The present invention relates to an accelerator and a particle therapy system including the accelerator.

### BACKGROUND ART

PTL 1 discloses a cyclotron including: a pair of magnetic poles which are provided in a pair with a beam closed orbit interposed therebetween, each of which has a plurality of convex portions and a plurality of concave portions arranged alternately in a circumferential direction, and each of which forms hill regions sandwiched between the convex portions and valley regions sandwiched between the concave portions along the closed orbit, a dee electrode provided in the valley region, and a radiofrequency wave generator which is disposed on an outer peripheral side in a radial direction of the beam closed orbit in at least one valley region other than the valley region provided with the dee electrode, and which generates a radiofrequency electric field for accelerating the beam.

Further, PTL 2 discloses, as an accelerator capable of efficiently extracting ion beams having different energies, an accelerator including a return yoke and a vacuum vessel in which an injection electrode is disposed closer to an entrance of a beam extraction path in the return yoke than a central axis of the vacuum vessel, magnetic poles are arranged radially from the injection electrode around the injection electrode in the return yoke, concave portions are arranged alternately with the magnetic poles in the circumferential direction of the return yoke, in the vacuum vessel, an orbital concentric region where a plurality of beam closed orbits centering on the injection electrode exist is formed, and an orbital eccentric region where a plurality of beam closed orbits decentered from the injection electrode exist is formed around the region, and in the orbital eccentric region, the beam closed orbits become dense between the injection electrode and the entrance of the beam extraction path, and the distance between the beam closed orbits becomes wide on the 180° opposite side of the entrance of the beam extraction path with the injection electrode as the base point.

### CITATION LIST

### Patent Literature

PTL 1: JP 2014-160613 A
PTL 2: International Publication No. 2016/092621

### SUMMARY OF INVENTION

### Technical Problem

High-energy nuclear beams used in particle therapy and physical experiments are generated using an accelerator.

There are several types of accelerators that obtain a beam with a kinetic energy of about 200 MeV per nucleon. Examples thereof include the cyclotron described in PTL 1 above, a synchrotron, a synchrocyclotron, and the variable energy accelerator described in PTL 2 above.

The feature of cyclotron and synchrocyclotron is that a beam orbiting in a static magnetic field is accelerated by a radiofrequency electric field. The beam increases its radius of curvature as it accelerates, travels to the outer orbits, and is extracted after reaching maximum energy. Therefore, the energy of the extracted beam is basically fixed.

In the synchrotron, the beam orbits on a fixed orbit by a temporal change of the magnetic field of the electromagnet that bends the beam and the frequency of the accelerating radiofrequency electric field. Therefore, it is possible to extract the beam before the maximum designed energy is reached, and the extraction energy is variable.

Like the cyclotron, the variable energy accelerator is characterized by decentering a beam orbit in one direction with acceleration while accelerating a beam orbiting in a magnetic field by the radiofrequency electric field.

The cyclotron described in PTL 1 and the variable energy accelerator described in PTL 2 are similar types of accelerators that accelerate a beam orbiting in a main magnetic field with a radiofrequency electric field. By making the average magnetic field on the orbit proportional to the relativistic y factor of the beam, the orbiting time is made constant regardless of energy. A main magnetic field distribution having this property is called an isochronous magnetic field. Now, under the isochronous magnetic field, the magnetic field is modulated along the orbit to secure the beam stability in the orbit plane and in the direction perpendicular to the orbit plane.

In order to achieve both the above-mentioned isochronism and beam stability, the main magnetic field distribution needs a maximum part (Hill) and a minimum part (Valley). The non-uniform magnetic field with this distribution can be formed by making the distance (gap) between the facing magnetic poles of the main magnet narrow in the Hill region and wide in the Valley region.

However, the difference between the Hill magnetic field and the Valley magnetic field is practically limited to about a saturation magnetic flux density of the magnetic pole material that is a ferromagnetic material. That is, the difference between the Hill magnetic field and the Valley magnetic field is limited to about 2 T.

On the other hand, when downsizing an accelerator that uses an isochronous magnetic field, it is necessary to increase the main magnetic field and reduce the deflection radius of the beam orbit. However, the difference between the main magnetic field and the above-mentioned Hill magnetic field and Valley magnetic field is proportional, and the above-mentioned limit is a factor that determines the practical size of the accelerator. Therefore, the cyclotron as described in PTL 1 and the variable energy accelerator as described in PTL 2, which are mentioned above, have a problem that downsizing is difficult.

The present invention provides a small-sized accelerator capable of extracting a beam of variable energy, and a particle therapy system including the accelerator.

### Solution to Problem

The present invention includes a plurality of means for solving the above problems, but if one example is given, it is an accelerator that accelerates a beam by a main magnetic field and a frequency-modulated radiofrequency electric field, the accelerator including: an acceleration radiofrequency acceleration system capable of frequency modulation and feeding an acceleration radiofrequency wave for accelerating the beam; an extraction radiofrequency acceleration system that feeds an extraction radiofrequency wave different in frequency from the acceleration radiofrequency wave for extracting a beam; a disturbance magnetic field region forming unit that forms a disturbance magnetic field region including a high-order magnetic field that includes a magnetic field component having a number of poles of two poles or more and that includes at least a quadrupole magnetic field component; and a magnetic shim and a septum magnet having a septum coil.

### Advantageous Effects of Invention

According to the present invention, it is possible to provide a small-sized accelerator capable of extracting a beam of variable energy. The problems, configurations, and effects other than those described above will be clarified from the description of the embodiments below.

### BRIEF DESCRIPTION OF DRAWINGS

FIG. 1 is a diagram showing an external appearance of a circular accelerator according to an embodiment of the present invention.

FIG. 2 is a diagram showing a cross-sectional structure of the circular accelerator of the embodiment.

FIG. 3 is a diagram showing a beam orbit of each energy in an orbit plane of the circular accelerator of the embodiment.

FIG. 4 is a diagram showing a cross-sectional structure of a radiofrequency kicker included in the circular accelerator of the embodiment.

FIG. 5 is a bird's-eye view of the radiofrequency kicker when viewed from an arrow B shown in FIG. 4.

FIG. 6 is a diagram showing an example of a cross section taken along a line AA′ in FIG. 2.

FIG. 7 is a magnetic field distribution diagram on a straight line r in FIG. 6.

FIG. 8 is a diagram showing another example of a cross section taken along the line AA′ of FIG. 2.

FIG. 9 is a view showing a cross-sectional structure of a septum magnet included in the circular accelerator of the embodiment.

FIG. 10 is a graph showing the relationship between the excitation current and the extraction beam energy of the septum coil that constitutes the septum magnet included in the circular accelerator of the embodiment.

FIG. 11 is a diagram showing another example of a cross section taken along the line AA′ of FIG. 2.

FIG. 12 is a diagram showing another example of a cross section taken along the line AA′ of FIG. 2.

FIG. 13 is a diagram showing an operation pattern of the circular accelerator of the embodiment.

FIG. 14 is a control system block diagram of an acceleration radiofrequency power supply, a radiofrequency kicker power supply, and a septum coil excitation power supply in the circular accelerator of the embodiment.

FIG. 15 is a diagram showing an overall configuration of a particle therapy system according to the embodiment of the present invention.

### DESCRIPTION OF EMBODIMENTS

Hereinafter, an embodiment of an accelerator of the present invention and a particle therapy system including the accelerator will be described with reference to the drawings. It should be noted that the followings are merely an embodiment and are not intended to limit the content of the invention to the following specific embodiment. The invention itself can be modified into various forms other than the following embodiment.

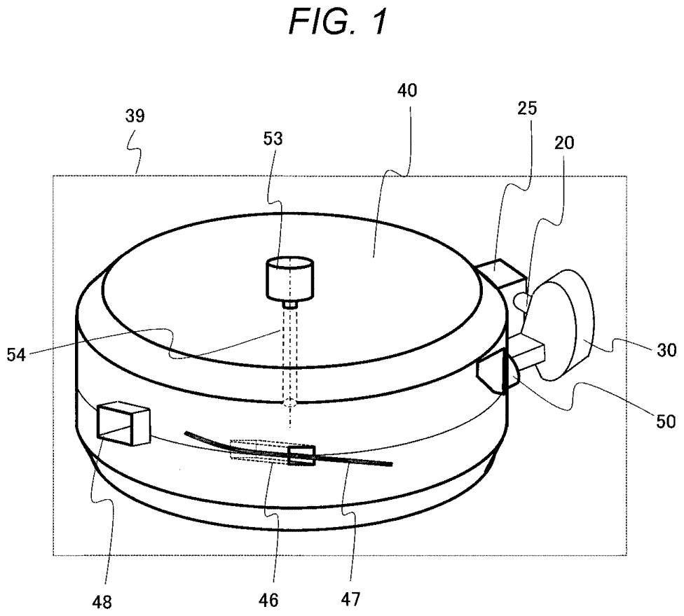

First, a structure of a circular accelerator according to a preferred embodiment of the present invention will be described with reference to FIGS. 1 to 14.

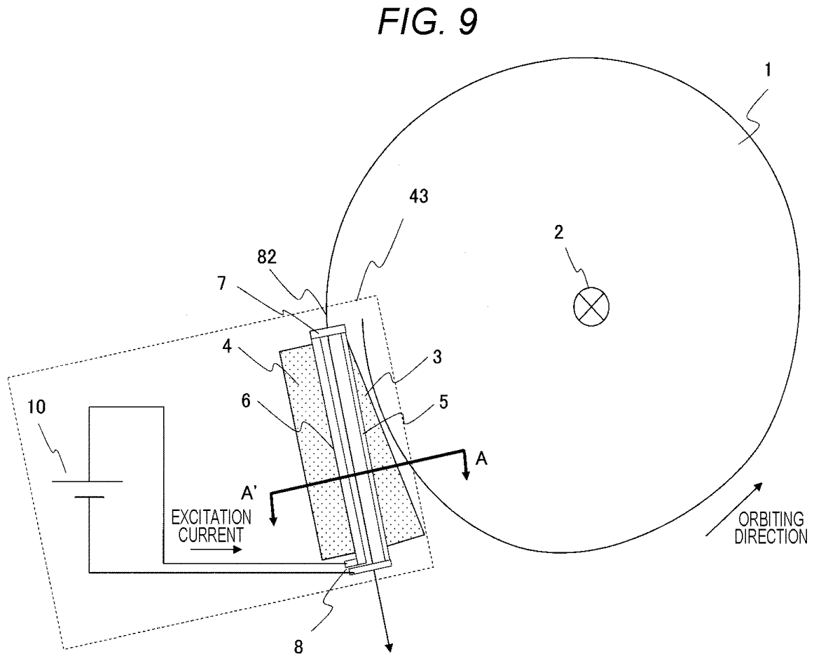

A circular accelerator 39 of this embodiment accelerates protons by a radiofrequency electric field obtained by temporally frequency-modulating a main magnetic field 2 of constant intensity (see FIG. 9), and the energy of the extracted beam is variable between 70 [MeV] and 235 [MeV].

The particles to be accelerated are not limited to protons, and heavy particle ions such as carbon and helium and electrons can be accelerated.

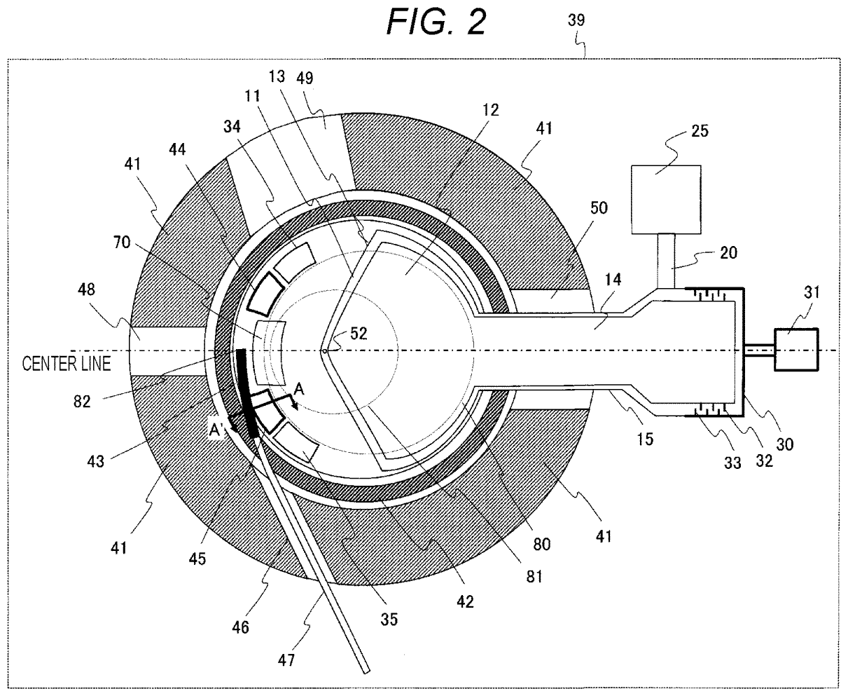

The external appearance of the circular accelerator 39 is shown in FIG. 1, and a cross-sectional structure is shown in FIG. 2.

As shown in FIG. 1, the circular accelerator 39 has an outer shell formed by a main magnet 40 that can be divided into upper and lower parts, and the inner side serving as a beam acceleration region is evacuated.

An input coupler 20 and a rotating condenser 30 are provided on the outer peripheral side of the circular accelerator 39. The circular accelerator 39 frequency-modulates the radiofrequency acceleration voltage using the rotating condenser 30.

An ion source 53 is installed above the main magnet 40, and the beam is injected into the circular accelerator 39 through a low energy beam transport 54. As the ion source 53, a microwave ion source, an ECR ion source, or the like can be applied. Note that the ion source may be disposed inside the vacuumed beam acceleration region inside the main magnet 40, and in that case, a PIG type ion source or the like can be applied.

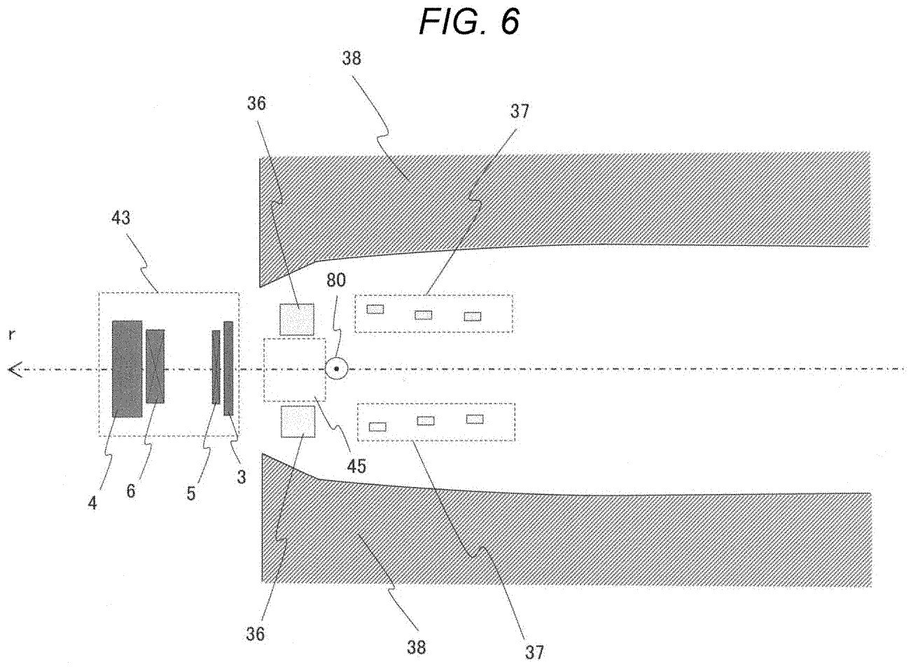

The main magnet 40 is composed of a main magnetic pole 38 (see FIG. 6 etc.), a return yoke 41, a main coil 42 and the like.

The return yoke 41 has a plurality of through holes, and among them, a beam through hole 46 for extracting the accelerated beam, a coil through hole 48 for drawing out an internal coil conductor to the outside, a vacuum drawing through hole 49, and a radiofrequency wave through hole 50 for radiofrequency acceleration cavity are provided on the connection surface of the upper and lower main magnets 40.

The radiofrequency acceleration cavity is a λ/2 resonance type cavity and includes a dee electrode 12, a dummy dee electrode 13, an outer conductor 15, an input coupler 20, a rotating condenser 30, and the like.

The rotating condenser 30 is a device for modulating the resonance frequency of the radiofrequency acceleration cavity, and has a fixed electrode 32 connected to the inner conductor 14, a rotating electrode 33 connected to the outer conductor 15, a motor 31, and the like. By driving the rotating condenser 30 with the motor 31, the area of the facing portion between the fixed electrode 32 and the rotating electrode 33 changes, so that the electrostatic capacitance changes and the resonance frequency of the radiofrequency acceleration cavity can be changed. As a result, a frequency-modulated acceleration voltage is generated in an acceleration gap 11 between the dee electrode 12 and the dummy dee electrode 13 to generate an acceleration radiofrequency wave for accelerating the beam.

The shape of the acceleration gap 11 shown in FIG. 2 shows the case where the number of harmonics is one, and is formed according to the shape of the beam orbit. Further, by changing the tip shape of the rotating electrode 33 or the fixed electrode 32, a resonance frequency modulation pattern suitable for beam acceleration can be obtained.

As shown in FIG. 2, an annular main coil 42 is installed inside the circular accelerator 39 along the inner wall of the return yoke 41. The main coil 42 is a superconducting coil in which a cryostat is installed around the coil, but a normal conducting coil can also be used. The main magnetic pole 38 is installed inside the main coil 42, and forms a magnetic field distribution suitable for beam circulation and extraction together with a trim coil (not shown) installed on the surface of the main magnetic pole 38.

A beam injection point 52 to be accelerated can be set near the center of the circular accelerator 39, but this embodiment shows a configuration in a case where the injection point 52 is shifted from the center of the circular accelerator 39 to the extraction side, and the beam orbit is made eccentric to the coil through hole 48 side.

A method for realizing such an eccentric orbit will be described.

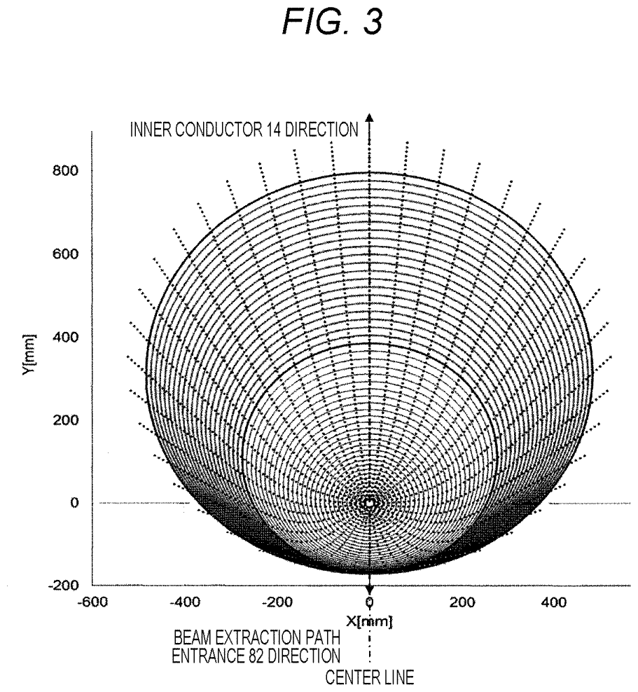

FIG. 3 shows the orbit of each energy. In FIG. 3, the closed orbits show orbits of 50 kinds of energy from the maximum energy of 235 [MeV] every 0.04 [Tm] of magnetic rigidity with a solid line. The dotted line is a line connecting the same orbiting phase of each orbit, and is called an equal orbiting phase line. The equal orbiting phase line is plotted for every orbiting phase π/20 from the aggregation region.

The acceleration gap 11 formed between the dee electrode 12 and the dummy dee electrode 13 facing the dee electrode 12 is installed along the equal orbiting phase line.

In FIG. 3, in the low energy region, an orbit centered around the ion injection point 52 is taken as in the cyclotron.

When further accelerated, the high-energy orbits are densely gathered in the vicinity of the septum magnet 43 used for extraction, and conversely, the orbits are in a positional relationship apart from each other in the vicinity where the inner conductor 14 is installed. The points where the orbits are densely gathered are called aggregation regions, and regions where the orbits are discrete are called discrete regions. By taking such an orbital arrangement and extracting the beam from the vicinity of the aggregation region, a beam kick amount required when extracting the beam can be reduced, so that the beam extraction of variable energy can be easily performed.

In order to generate an orbit structure and a stable oscillation around the orbit as described above, the circular accelerator 39 of the present embodiment uses a main magnetic field distribution in which the value of the magnetic field becomes smaller toward the radially outer peripheral side of the designed orbit. Also, the magnetic field is constant along the designed orbit. Therefore, the designed orbit becomes circular, and the orbit radius and the orbiting time increase as the beam energy increases.

The designed orbit of the circular accelerator 39 of this embodiment will be described below based on Equation (1).

$$\begin{matrix}[ Math . 1 ] & \\ n = - \frac{ρ}{\vert B \vert} \frac{\partial B}{\partial r} & ( 1 )\end{matrix}$$

Here, ρ represents a deflection radius of the designed orbit, B represents a magnetic field strength, and δB/δr represents a radial magnetic field gradient.

By setting a normalized magnetic field gradient n defined by Equation (1) to be greater than zero and less than one, particles that are slightly displaced from the designed orbit in the radial direction receive a restoring force to restore the particles to the designed orbit, and at the same time, particles displaced in the direction perpendicular to the orbit plane also receive a restoring force from the main magnetic field 2 in the direction of restoring the particles to the orbit plane.

That is, if the magnetic field is appropriately reduced with respect to the energy of the beam, the particles that are displaced from the designed orbit will always receive a restoring force in the direction of restoring the particles to the designed orbit and oscillate near the designed orbit. This makes it possible to make the beam stably orbit and accelerate the beam. In addition, the betatron oscillation frequency (horizontal tune) in the direction parallel to the orbit plane is set to a value less than one and close to one in the beam of full energy.

As described above, the above-mentioned main magnetic field distribution is excited by feeding a predetermined excitation current through the main coil 42 and the trim coil. The shape of the main magnetic pole 38 is symmetrical with respect to the orbit plane, and has only a magnetic field component in the direction perpendicular to the orbit plane on the orbit plane.

In the circular accelerator 39 of this embodiment, the main magnetic field 2 is a weakly converging magnetic field. Therefore, the main magnetic field 2 can be increased without being restricted by the Hill magnetic field and the Valley magnetic field in an AVF (Azimuthally Varing Field) cyclotron with an isochronous magnetic field, so that the deflection radius of the beam orbit can be reduced.

The AVF cyclotron is a cyclotron of a system for synchronizing the acceleration frequency with the rotation frequency of the accelerating particle by shortening the closed orbit length of the particle by making the strength of the magnetic field stronger as the radius increases and shortening the rotation cycle.

Next, a method for extracting the beam will be described. In the circular accelerator 39 of this embodiment, for extracting the beam, the radiofrequency kicker 70, the peeler magnetic field region 44, the regenerator magnetic field region 45, the septum magnet 43, the upstream coil 34, the downstream coil 35, and the high energy beam transport 47 are used.

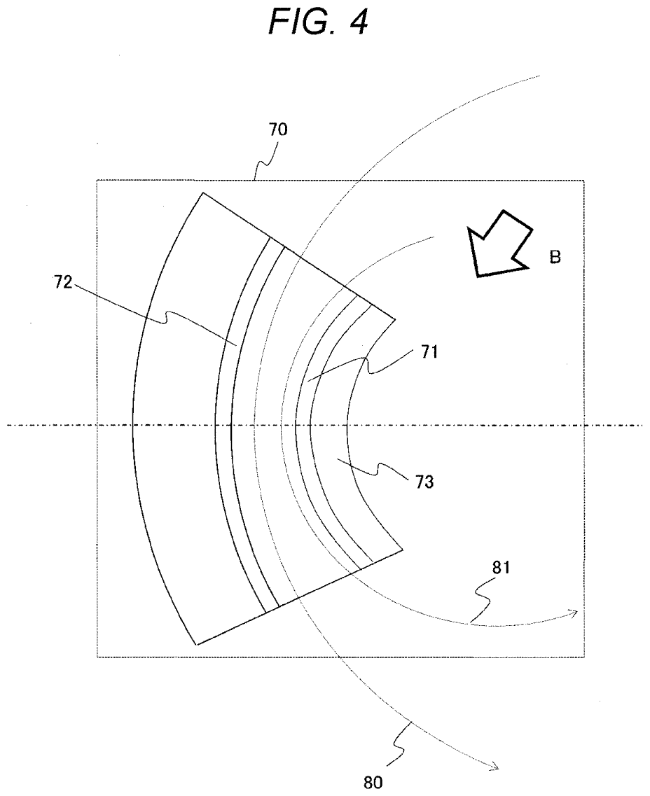

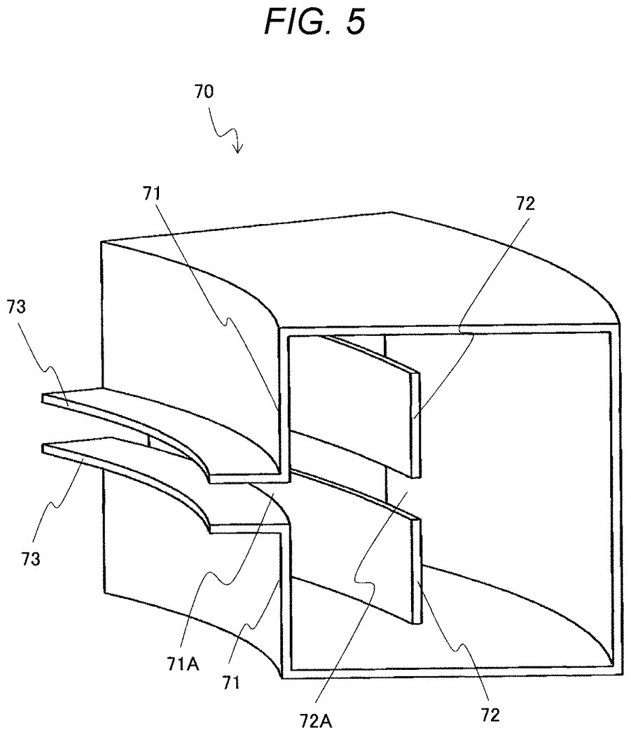

A cross-sectional structure of the radiofrequency kicker 70 is shown in FIG. 4. Further, FIG. 5 shows a bird's-eye view of the radiofrequency kicker 70 seen from the direction B in FIG. 4.

The radiofrequency kicker 70 is a device for feeding an extraction radiofrequency wave for extracting a beam, and includes a ground electrode 71, a high-voltage electrode 72, and the like. The extraction radiofrequency wave is different in frequency from the acceleration radiofrequency wave.

As shown in FIG. 4, the ground electrode 71 and the high-voltage electrode 72 are installed facing each other so as to sandwich the maximum extraction energy orbit 80 and the minimum extraction energy orbit 81, and the shapes are determined so that a radiofrequency electric field acts in a direction orthogonal to the orbit in the orbit plane.

Further, as shown in FIGS. 4 and 5, a metal projection 73 is attached to the ground electrode 71 so as to increase the concentration of the radiofrequency electric field generated between the ground electrode 71 and the high-voltage electrode 72.

The high-voltage electrode 72 to which a radiofrequency voltage is fed insulating supports the ground electrode 71. The method of insulating support is not particularly limited, and a method of supporting with an insulating support (not shown) or the like can be considered. In addition, the ground electrode 71 and the high-voltage electrode 72 have a cooling mechanism (not shown) against heat generation due to radiofrequency energization.

Both the ground electrode 71 and the high-voltage electrode 72 have passage openings 71A, 72A respectively near the orbit plane through which the beam passes. These passage openings 71A, 72A are set to a size that does not cause a beam collision in consideration of the spread due to betatron oscillation in the direction perpendicular to the beam orbit plane.

The radiofrequency kicker 70 of this embodiment has a shape in which the end faces of the beam entrance side and the beam exit side are open as shown in FIG. 4, but the end faces may be closed by the ground electrode 71 except for the beam passage opening 71A to form a cavity resonator structure.

The radiofrequency kicker 70 may be disposed so that the electric field acts on both the minimum extraction energy orbit 81 and the maximum extraction energy orbit 80. However, it is preferable to dispose it in the vicinity of the beam extraction path entrance 82 as shown in FIG. 2.

Returning to FIG. 2, the peeler magnetic field region 44 and the regenerator magnetic field region 45 are regions where a multipole magnetic field (disturbance magnetic field) acting on the beam exists. This multipole magnetic field includes a high-order magnetic field that includes a magnetic field component having a number of poles of two poles or more and that includes at least a quadrupole magnetic field component. A multipole magnetic field with four or more poles or a two-pole magnetic field may be included.

The peeler magnetic field region 44 has a magnetic field gradient in a direction that weakens the main magnetic field 2 toward the outer peripheral side in the radial direction. On the other hand, the regenerator magnetic field region 45 has a magnetic field gradient in the direction that reversely strengthens the main magnetic field 2 toward the outer peripheral side in the radial direction. As the peeler magnetic field region 44, a region where the main magnetic field 2 at the magnetic pole end portion decreases can also be used.

The peeler magnetic field region 44 and the regenerator magnetic field region 45 are arranged on the outer peripheral side of the maximum extraction energy orbit 80 and in an azimuthal region sandwiching the beam extraction path entrance 82, respectively. However, the peeler magnetic field region 44 is disposed on the upstream side with respect to the beam traveling direction, and the regenerator magnetic field region 45 is disposed on the downstream side.

The peeler magnetic field region 44 and the regenerator magnetic field region 45 are formed by fixedly disposing a plurality of magnetic pole pieces and/or coils made of a magnetic material with respect to the main magnetic pole 38 with a non-magnetic material.

When the magnetic pole pieces and the coils are used together, the coils may be arranged in a space different from the peeler magnetic field region 44 and the regenerator magnetic field region 45 in which the magnetic pole pieces are arranged. FIG. 2 shows an example of such an arrangement. That is, the magnetic pole pieces are arranged in or around the peeler magnetic field region 44 and the regenerator magnetic field region 45, respectively. For example, the upstream coil 34 and the downstream coil 35 are arranged as shown in FIG. 2.

Whichever location they are arranged, the upstream coil 34 generates a magnetic field in a direction that weakens the main magnetic field 2 and the downstream coil 35 generates a magnetic field in a direction that strengthens the main magnetic field 2.

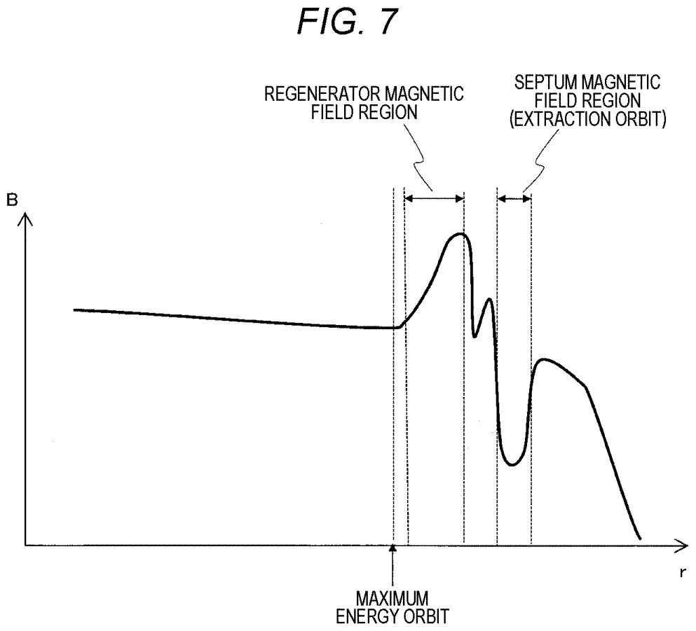

FIG. 6, which is a view taken along the arrow AA′ in FIG. 2, shows an example of the arrangement of magnetic pole pieces in the regenerator magnetic field region 45 when the upstream coil and the downstream coil are not used. Further, FIG. 7 shows a magnetic field distribution diagram on the straight line r in FIG. 6.

As shown in FIG. 6, as the magnetic pole pieces, a magnetic field gradient shim 36 for generating a magnetic field gradient in the regenerator magnetic field region 45, and a magnetic field correction shim 37 for canceling the unnecessary leakage magnetic field that is generated by the magnetic field gradient shim 36 on the inner peripheral side of the maximum extraction energy orbit 80 are used. The main magnetic field 2 on the r-axis in FIG. 6 has a distribution as shown in FIG. 7, and the beam stably orbits up to the maximum extraction energy orbit.

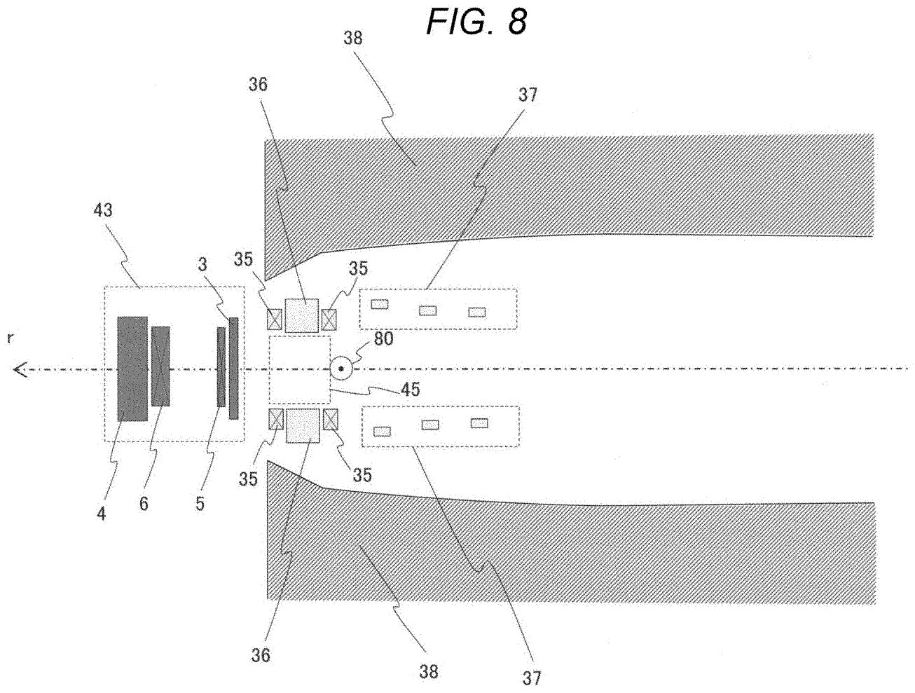

Further, FIG. 8 shows a case where an upstream coil and a downstream coil are used. As shown in FIG. 8, when the downstream coil 35 is arranged in the regenerator magnetic field region 45, the downstream coil 35 is wound around the magnetic field gradient shim 36 which is a magnetic pole piece. For the peeler magnetic field region 44, the upstream coil 34 is wound around the magnetic field gradient shim (not shown).

As shown in FIG. 9, the septum magnet 43 includes an inner peripheral side shim 3 of a magnetic material, an outer peripheral side shim 4 of a magnetic material, a septum coil that supplies bipolar current, and a bipolar power supply 10. The septum coil is composed of an inner peripheral side septum coil conductor 5, an outer peripheral side septum coil conductor 6, a coil conductor connecting portion 7 and a coil lead-out portion 8.

The septum magnet 43 is disposed on the downstream side of the beam extraction path entrance 82. FIG. 9 shows the case where the septum coil is constructed with one turn. That is, the inner peripheral side septum coil conductor 5 and the outer peripheral side septum coil conductor 6 are electrically connected by the coil conductor connecting portion 7, and are electrically connected to the bipolar power supply 10 for coil excitation at the coil lead-out portion 8.

The coil conductor connecting portion 7 and the coil lead-out portion 8 may be provided in reverse, and the coil lead-out portion 8 may be provided on the side closer to the beam extraction path entrance 82 and the coil conductor connecting portion 7 may be provided on the opposite side. Further, as shown in FIG. 8, the coil lead-out portion 8 does not need to be provided at the end portion of the inner peripheral side septum coil conductor 5 or the outer peripheral side septum coil conductor 6, and may be provided in the middle portion in the beam extraction direction with a part of the inner peripheral side septum coil conductor 5 or the outer peripheral side septum coil conductor 6 cut.

By feeding an excitation current to the septum coil by the bipolar power supply 10, it is possible to form a bipolar magnetic field inside the septum magnet 43. Each of the inner peripheral side septum coil conductor 5, the outer peripheral side septum coil conductor 6, the coil conductor connecting portion 7, and the coil lead-out portion 8 has a cooling means against heat generation, and is supported by a support (not shown) so that the deformation due to the electromagnetic stress caused by the excitation current is within an allowable range.

The inner peripheral side shim 3 and the outer peripheral side shim 4 are magnetic and made of, for example, laminated steel plates.

As shown in FIG. 9, the inner peripheral side shim 3 has a wedge shape so as not to interfere with an orbit 1 of the last one turn immediately before the beam reaches the beam extraction path entrance 82. The outer peripheral side shim 4 may be installed so as to face the inner peripheral side shim 3 with the beam passing region interposed therebetween, and the shape thereof is not particularly limited.

Here, in the extraction energy band, the Bρ product of the maximum extraction energy is Bρₘₐₓ, the Bρ product of the minimum extraction energy is Bρₘᵢₙ, and the beam energy corresponding to the Bρ product equal to (Bρₘₐₓ+Bρₘᵢₙ)/2 is defined as the intermediate energy. Then, the thickness and shape of each shim are set so that only the inner peripheral side shim 3 and the outer peripheral side shim 4 form a magnetic field for extracting the beam of intermediate energy.

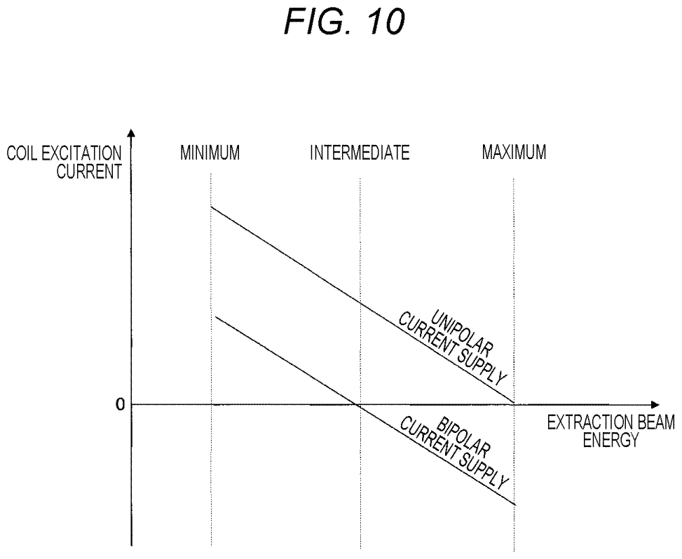

In the septum magnet 43 designed in this way, when extracting a beam of an energy different from the intermediate energy, an appropriate excitation current is supplied to the septum coil. FIG. 10 shows the relationship between the excitation current of the septum coil and the extraction beam energy.

As shown in FIG. 10, in a case where a beam of an energy lower than the intermediate energy is extracted, the case is coped with by feeding an excitation current having a polarity opposite to that in a case where a beam of a high energy is extracted to the septum coil.

Since the circular accelerator 39 of this embodiment extracts a pulse, the bipolar power supply 10 that is the excitation power supply of the septum coil can also reduce the power consumption of the power supply by pulse excitation instead of DC excitation. In this case, the number of turns of the septum coil is preferably ten turns or less in order to suppress inductance.

A unipolar power supply can be used instead of the bipolar power supply 10. In this case, as shown in FIG. 10, as for the thickness of the inner peripheral side shim 3 and the outer peripheral side shim 4, it is desirable to set the thickness and shape of each shim so that only the inner peripheral side shim 3 and the outer peripheral side shim 4 form a magnetic field in which a beam of the maximum energy is extracted. Moreover, it is desirable to extract beams other than the beam of the maximum energy by feeding a current to the septum coil.

The cross section taken along the line AA′ in FIG. 9 (the same as the cross section taken along the line AA′ in FIG. 2) is shown in FIGS. 6, 8, 11, and 12.

As shown in FIGS. 6 and 8, the inner peripheral side shim 3 and the outer peripheral side shim 4 can be placed independently without being connected to each other.

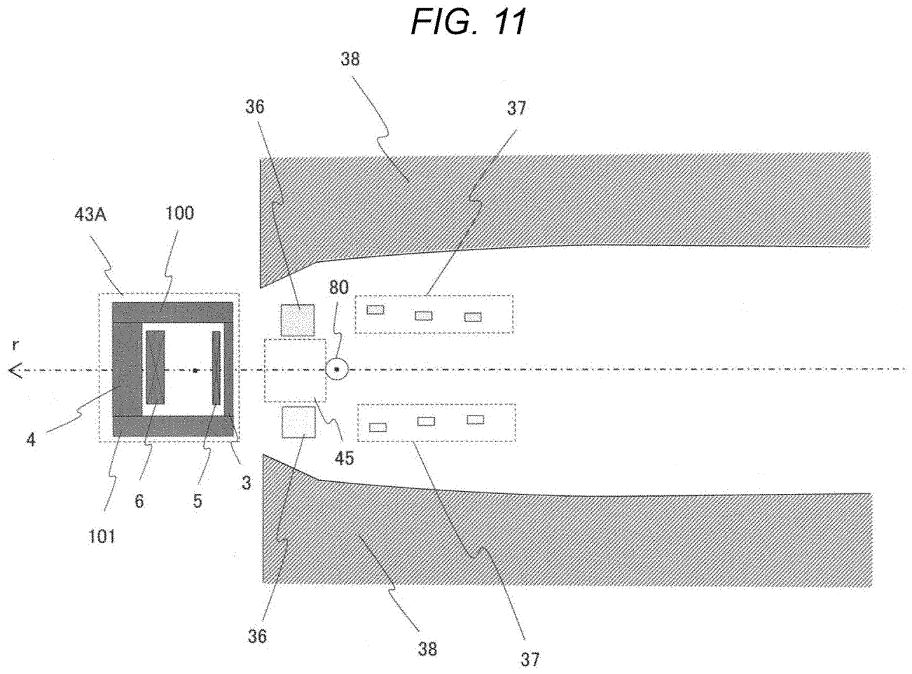

Further, as shown in FIG. 11, it is possible to use a septum magnet 43A having a structure in which the inner peripheral side shim 3 and the outer peripheral side shim 4 are connected on the upper surface side by an upper shim 100 disposed on the upper side in the vertical direction with respect to the beam orbit plane, and are connected on the lower surface side by a lower shim 101 disposed on the lower side in the vertical direction with respect to the beam orbit plane.

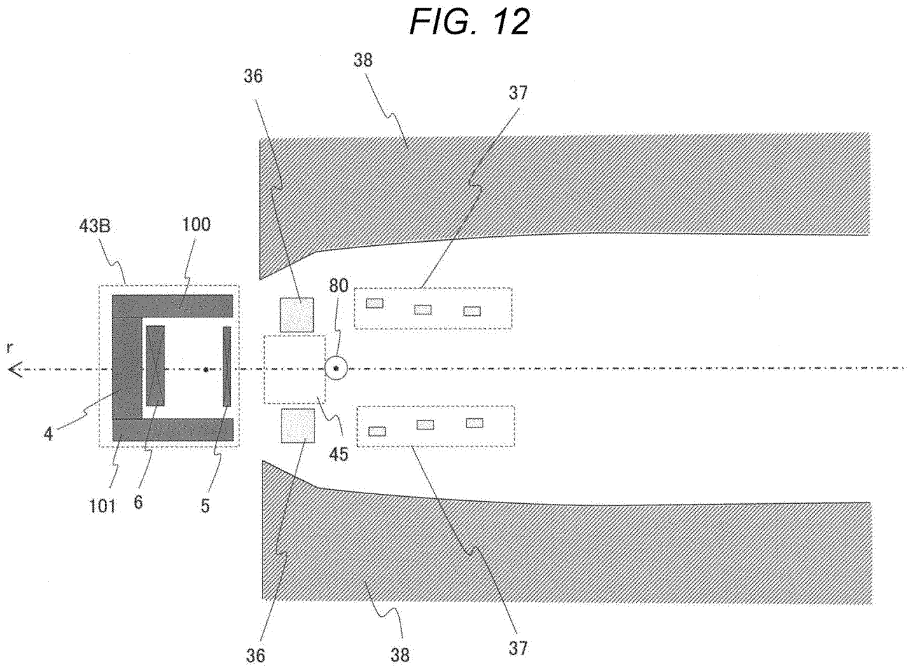

Further, as shown in FIG. 12, in order to avoid interference with the beam orbit, a septum magnet 43B having a structure in which the inner peripheral side shim 3 is omitted may be used. Although FIG. 12 shows the case where the upper shim 100 and the lower shim 101 are arranged, the upper shim 100 and the lower shim 101 can be appropriately omitted as in FIG. 6 and the like.

Further, it is possible to use a structure in which the inner peripheral side shim 3 is divided in the vicinity of the orbit plane, so that the interference with the beam orbit can be more reliably suppressed. Even when the inner peripheral side shim 3 has a divided structure, the shim made of a magnetic material can be appropriately disposed at the same position as the upper shim 100 or the lower shim 101.

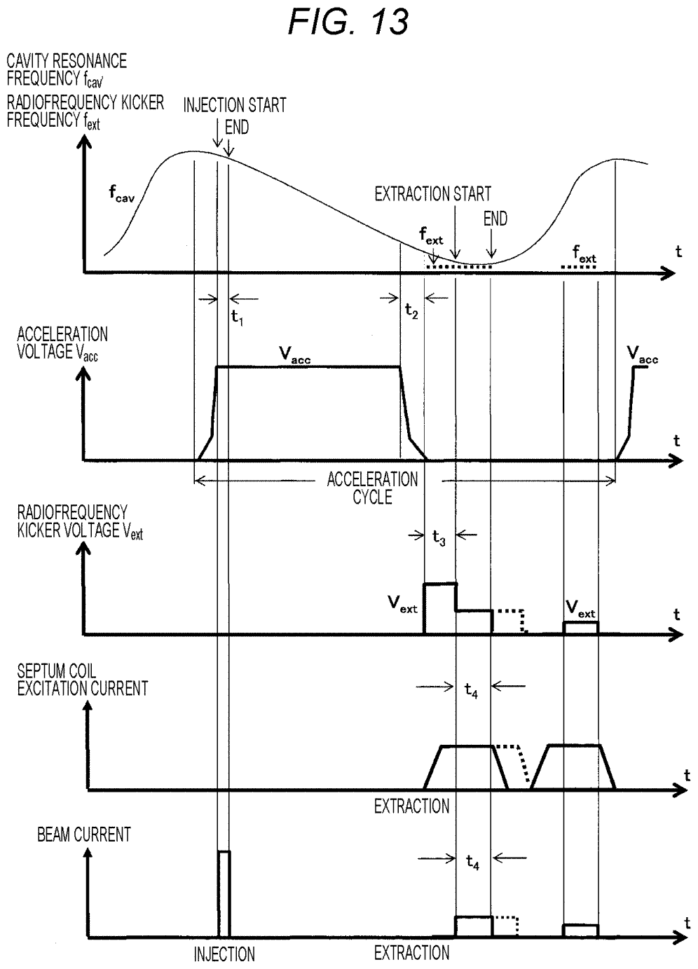

Next, a beam extraction procedure will be described with reference to FIG. 13.

One acceleration cycle starts when the acceleration radiofrequency wave rises, that is, when the feeding of the acceleration voltage V_{acc} is started at the timing when the resonance frequency f_{cav} of the radiofrequency acceleration cavity reaches a predetermined value.

After V_{acc} rises, the beam is injected into the vacuum space inside the main magnetic pole 38 from the ion source 53, and the radiofrequency wave capture of the beam ends after the time t₁ has elapsed.

When the captured beam is accelerated to reach the desired extraction energy, a control signal for cutting off the acceleration radiofrequency wave is issued.

Then, when the time t₂ has elapsed, the acceleration radiofrequency wave is turned off. At the same time, feeding of the radiofrequency voltage Vₑₓₜ to the radiofrequency kicker 70 is started. If the radiofrequency kicker 70 is designed not to have a resonator structure but to have an appropriate capacitance, the radiofrequency voltage of the radiofrequency kicker 70 quickly rises with a response of several μs or less.

The frequency fₑₓₜ of the extraction radiofrequency voltage Vₑₓₜ is set to be equal to the product Δνᵣ×fᵣₑᵥ of the fractional part Δνᵣ of the horizontal tune νᵣ of the orbiting beam and the orbiting frequency fᵣₑᵥ. As a result, the amplitude of the horizontal betatron oscillation continues to increase.

Since the ground electrode 71 and the high-voltage electrode 72 are shaped so that a radiofrequency electric field acts in a direction (horizontal direction) orthogonal to the orbit in the orbit plane, the beam is kicked by this radiofrequency electric field, and thereby efficiently increasing the amplitude of the betatron oscillation in the horizontal direction. However, only by the radiofrequency kicker 70, turn separation sufficient to extract the beam cannot be obtained. Therefore, the peeler magnetic field region 44 and the regenerator magnetic field region 45 are required.

The beam eventually reaches the peeler magnetic field region 44 and the regenerator magnetic field region 45 by the action of the radiofrequency kicker 70. The beam is kicked to the outer peripheral side when passing through the peeler magnetic field region 44, and is kicked to the inner peripheral side when passing through the regenerator magnetic field region 45. At this time, horizontal tune≈1, and the peeler magnetic field region 44 and the regenerator magnetic field region 45 both have an appropriate magnetic field gradient in the radial direction, so the kick amount gradually increases while the beam orbits a plurality of times to increase the turn separation. In other words, the turn separation can be exponentially increased by using the resonance condition of the betatron oscillation of 2νᵣ=2.

Since the septum magnet 43 is installed at the beam extraction path entrance 82, when a turn separation that is far beyond the total thickness of the inner peripheral side shim 3 and the inner peripheral side septum coil conductor 5 is obtained, the beam is guided to the inside of the septum magnet 43.

At this time, if the excitation current of the septum coil has an appropriate value according to the beam energy, the beam is sufficiently bent to be guided to the high energy beam transport 47 in the subsequent stage, and beam extraction is started. FIG. 13 shows an example in which the septum coil is pulse-excited.

Before the beam extraction is started, supply of the excitation current of the septum magnet 43 is started. Since the beam extraction is not performed after the feeding of extraction radiofrequency wave is ended, it is desirable to cut off the excitation current of the septum coil, but if the time interval until the next beam extraction is short, the excitation of the septum coil may be continued.

Note that, as shown in FIG. 13, immediately after the feeding of the radiofrequency voltage to the radiofrequency kicker 70 is started, the highest possible radiofrequency voltage (Vₑₓₜ) is fed and the amplitude of Vₑₓₜ can be reduced immediately before the beam reaches the peeler magnetic field region 44 and the regenerator magnetic field region 45. As a result, the time until the start of beam extraction can be shortened and the dose rate can be improved.

The beam extraction current can be adjusted by controlling the amplitude of Vₑₓₜ after the beam has reached the peeler magnetic field region 44 and the regenerator magnetic field region 45. That is, as the amplitude of Vₑₓₜ increases, the beam extraction current also increases. Further, the beam extraction can be stopped by stopping the feeding of Vₑₓₜ at an arbitrary timing. Therefore, it is possible to irradiate the spot dose required for scanning irradiation with just one extraction pulse beam and in just proportion, and the dose rate is improved.

Further, it is possible to adjust the beam extraction current instead of controlling the amplitude of Vₑₓₜ, but by controlling one or more of sweeping the frequency of Vₑₓₜ or changing the phase.

Further, if the orbital charge remains in the accelerator after the extraction, the beam extraction can be restarted by feeding Vₑₓₜ again, and thus the charge can be used for the next spot irradiation. For this reason, the charge injected from the ion source 53 can be used without waste, and the dose rate is further improved. It should be noted that one acceleration cycle ends when the amount of orbital charge remaining in the accelerator falls below a certain level. The beam is extracted by repeating such an acceleration cycle.

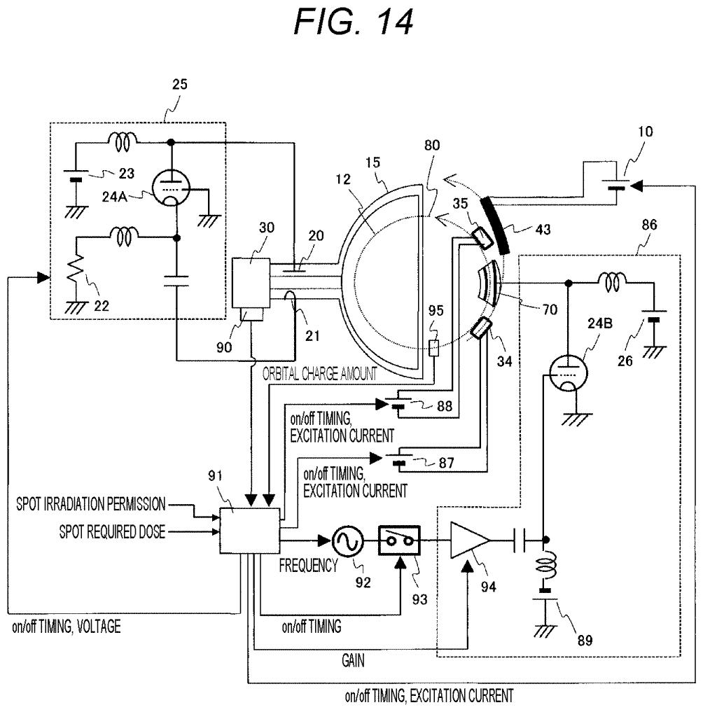

FIG. 14 shows a block diagram of the radiofrequency power supply and control system that realize the above extraction method. FIG. 14 shows a configuration in which triodes 24A and 24B are used for both an acceleration radiofrequency power supply 25 and a radiofrequency kicker power supply 86, but quadrupole tubes or semiconductor amplifiers can be used in addition.

As the beam accelerating system, an input coupler 20, a pickup loop 21, an acceleration radiofrequency power supply 25 having a cathode resistance 22, a plate DC power supply 23, and a triode 24A, a rotating condenser 30, an angle detection mechanism 90, a dee electrode 12, and an outer conductor 15 are used.

The acceleration radiofrequency power supply 25 is of a self-oscillation type, and is a system of feeding back a part of the acceleration radiofrequency wave to the cathode circuit by the pickup loop 21. The radiofrequency acceleration voltage is controlled by modulating the output voltage of the plate DC power supply 23 at high speed. The cathode bias potential is applied by dividing the plate potential by the cathode resistance 22 as shown in FIG. 14, or by using a cathode power supply. The acceleration radiofrequency power supply 25 may be of a separately excited oscillation type, the pickup loop 21 may be omitted, and a pre-programming type original oscillator output that has been pre-amplified may be used as the input of the triode 24A.

For the beam extraction system, a bipolar power supply 10, a septum magnet 43, an upstream coil 34, a downstream coil 35, an upstream coil power supply 87, a downstream coil power supply 88, a triode 24B, a plate DC power supply 26, a grid bias power supply 89, an original oscillator 92, a switch 93, a preamplifier 94, the radiofrequency kicker power supply 86, and the radiofrequency kicker 70 are used.

The original oscillator 92 generates a signal in a certain frequency band for the radiofrequency kicker 70. It is assumed that the signal includes a necessary frequency band component in consideration of the tune spread of the beam and the fluctuation of the horizontal tune during the feeding of the radiofrequency voltage Vₑₓₜ to the radiofrequency kicker 70. This signal is amplified by the preamplifier 94 via the switch 93. After amplification, it is further amplified by the triode 24B and supplied to the radiofrequency kicker 70. The amplitude of the radiofrequency voltage Vₑₓₜ of the radiofrequency kicker 70 is controlled by changing the gain of the preamplifier 94 or modulating the output voltage of the plate DC power supply 26 at high speed.

An arithmetic unit 91 controls the feeding timing of the acceleration radiofrequency wave f_{cav} in the acceleration system and the feeding timing of the extraction radiofrequency wave fₑₓₜ in the beam extraction system.

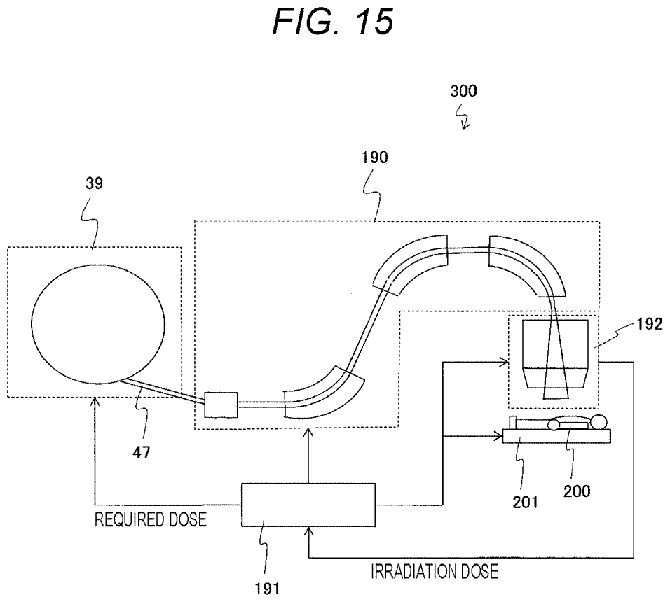

The arithmetic unit 91 receives input of information on the frequency modulation pattern of the acceleration radiofrequency wave f_{cav} detected from the angle detection mechanism 90 of the rotating condenser 30 or the pickup signal of the acceleration radiofrequency wave, the permission of each spot irradiation from the control system 191 (see FIG. 15), and the required dose to each spot, and outputs a command signal of ON/OFF timing and the voltage amplitude of the acceleration radiofrequency wave f_{cav} to the acceleration radiofrequency power supply 25.

Further, the arithmetic unit 91 outputs a command signal of ON/OFF timing and the excitation current of the septum magnet 43 to the bipolar power supply 10 based on the input of the above information.

Further, the arithmetic unit 91 outputs a command signal of ON/OFF timing and the amplitude of the voltage Vₑₓₜ of the radiofrequency kicker 70 to the radiofrequency kicker power supply 86.

Further, the arithmetic unit 91 outputs a command signal of on/off timing and an excitation current to the downstream coil power supply 88, that is, to the downstream coil 35, and outputs a command signal of on/off timing and an excitation current to the upstream coil power supply 87, that is, to the upstream coil 34.

Further, a beam monitor 95 that electrostatically or magnetically measures the amount of orbital charges remaining inside the accelerator for the beams in all extraction energy bands is installed at any arbitrary place on the beam orbit. Then, when the amount of orbital charge decreases below a certain level, the arithmetic unit 91 restarts the feeding of the acceleration voltage, and repeats the processes of capturing, accelerating and extracting.

Next, the overall configuration when the circular accelerator 39 of the present embodiment described above is applied to a particle therapy system used for particle therapy and the like will be described using FIG. 15. FIG. 15 is a diagram showing the overall configuration of the particle therapy system of this embodiment.

In FIG. 15, a particle therapy system 300 includes a circular accelerator 39, a high energy beam transport 47, a rotating gantry 190, an irradiation system 192, a treatment table 201, and a control system 191.

The ion beam of specific energy extracted from the circular accelerator 39 is transported to the irradiation system 192 by the high energy beam transport 47 and the rotating gantry 190. The transported ion beam of specific energy is shaped by the irradiation system 192 so as to match the target shape, and is irradiated with a predetermined amount on the target volume of the patient 200 lying on the treatment table 201.

The control system 191 executes the operations of the circular accelerator 39, the high energy beam transport 47, the rotating gantry 190, the irradiation system 192, and the treatment table 201.

The control system 191 is composed of a computer or the like. The computer constituting these includes a CPU, a memory, an interface, etc., and control of the operation of each device and various arithmetic processes described later are executed based on various programs. These programs are stored in an internal recording medium, an external recording medium, or a data server in each component, and are read and executed by the CPU.

The operation control process may be integrated into one program, may be divided into a plurality of programs, or may be a combination thereof. Further, part or all of the program may be realized by dedicated hardware or may be modularized. Furthermore, various programs may be installed in each device from a program distribution server, an internal storage medium, or an external recording medium.

At this time, since the circular accelerator 39 of the present invention can be downsized and the beam loss is reduced as described above, the dose rate is improved, the irradiation time is shortened, and the patient throughput can be increased.

The beam can be directly extracted from the circular accelerator 39 to the irradiation system 192. Further, a plurality of irradiation systems 192 can be provided. Further, the irradiation system 192 may be fixed without rotating. The irradiation method used in the irradiation system 192 is not particularly limited, and either a scanning method of scanning a beam or a wobbler method using a scatterer may be used.

Next, the effect of this embodiment will be described.

The particle therapy system 300 according to the present embodiment described above includes the circular accelerator 39 that accelerates the beam by the main magnetic field 2 and the frequency-modulated radiofrequency electric field, and the irradiation system 192 that irradiates the beam of specific energy extracted from the circular accelerator 39. Among them, the circular accelerator 39 includes the acceleration radiofrequency acceleration system capable of frequency modulation and feeding an acceleration radiofrequency wave for accelerating the beam; the radiofrequency kicker 70 that feeds an extraction radiofrequency wave different in frequency from the acceleration radiofrequency wave for extracting a beam; the peeler magnetic field region 44 and the regenerator magnetic field region 45 for forming a disturbance magnetic field region including a high-order magnetic field that includes a magnetic field component having a number of poles of two poles or more and that includes at least a quadrupole magnetic field component; and the magnetic shim and the septum magnet 43, 43A, 43B having the inner peripheral side septum coil conductor 5 and the outer peripheral side septum coil conductor 6.

With the above-described configuration, it is possible to achieve downsizing and extract a beam of variable energy in a circular accelerator for increasing the main magnetic field 2 for downsizing. Further, since the variable energy beam can be extracted without using a scatterer, the beam current value lost during extraction can be minimized, and a high irradiation dose rate can be realized. Furthermore, since the extraction energy can be changed electrically, there is also an advantage that the time required for energy switching is shorter than that in the method of mechanically moving the scatterer.

Such a circular accelerator 39 greatly contributes to improving the patient throughput of the particle therapy system.

Further, since the septum magnet 43, 43A, 43B further has a bipolar power supply 10 for supplying bipolar current to the septum coil, the excitation current amplitude can be approximately halved as compared with the case where bipolar current is not supplied. Therefore, the heat load on the septum coil can be reduced to about ¼. Therefore, since the structure of the septum magnet 43, 43A, 43B can be simplified, downsizing and cost reduction can be achieved.

Further, the shim is constructed by the outer peripheral side shim 4 disposed on the outer peripheral side of the beam closed orbit with respect to the outer peripheral side septum coil conductor 6, so that the magnetic field to be generated by the septum coil can be reduced, and the heat load and electromagnetic stress of the septum coil can be suppressed.

Further, the shim is constructed by an inner peripheral side shim 3 disposed on the inner peripheral side of the beam closed orbit with respect to the inner peripheral side septum coil conductor 5, and an outer peripheral side shim 4 disposed on the outer peripheral side of the beam closed orbit with respect to the outer peripheral side septum coil conductor 6, so that it is also possible to reduce the magnetic field to be generated by the septum coil, and to suppress the heat load and electromagnetic stress of the septum coil.

Furthermore, the inner peripheral side shim 3 has a wedge shape that does not interfere with the beam closed orbit, so that the beam loss in the circular accelerator 39 can be suppressed and a higher irradiation dose rate can be realized.

Further, since the inner peripheral side shim 3 and the outer peripheral side shim 4 are independently arranged without being connected to each other, the septum magnet 43 can be constructed with a simple structure, thus achieving further downsizing and cost reduction.

Further, the upper shim 100 disposed vertically above the beam orbit plane and the lower shim 101 disposed vertically below the beam orbit plane are further provided. At least one of the inner peripheral side shim 3 and the outer peripheral side shim 4 is connected to the upper shim 100 and the lower shim 101, so that a magnetic field for guiding the beam to the high energy beam transport 47 generated by the septum magnet 43A, 43B can more efficiently shield the end leakage magnetic field of the main magnetic field 2 formed by the main magnet 40 and reduce the excitation current of the septum coil.

Further, the shim is a laminated steel plate core, and by configuring the coil winding composed of the inner peripheral side septum coil conductor 5 and the outer peripheral side septum coil conductor 6 with ten turns or less, it becomes possible to perform pulse excitation and suppress the power consumption of the excitation power supply.

Further, by forming the distribution of the main magnetic field 2 so that the beam injection point 52 is shifted from the center of the circular accelerator 39 to the extraction side, it is possible to form the aggregation region where the beam closed orbits are dense on the side near the beam extraction path entrance 82. For this reason, the beam kick amount of the radiofrequency kicker 70 required when extracting a beam of variable energy is smaller than that when the beam injection point 52 is placed at the center of the circular accelerator 39 and the main magnetic field distribution is formed so as to have a concentric orbit with respect to this center, so that the radiofrequency power required for the radiofrequency kicker can be suppressed low.

Further, the peeler magnetic field region 44 and the regenerator magnetic field region 45 are arranged at one place each, and the peeler magnetic field region 44 is used as a first disturbance magnetic field region having a magnetic field gradient in which the main magnetic field 2 weakens toward the outer peripheral side in the radial direction, and the regenerator magnetic field region 45 is used as a second disturbance magnetic field region having a magnetic field gradient in which the main magnetic field 2 is strengthened toward the outer peripheral side in the radial direction, so that the beam that has reached these disturbance magnetic field regions by the kick by the radiofrequency kicker 70 is kicked further to enter the entrance of the septum magnet 43, and is eventually taken out of the accelerator.

Furthermore, if the peeler magnetic field region 44 and the regenerator magnetic field region 45 are formed only by the magnetic field gradient shim 36 and the magnetic field correction shim 37 made of a magnetic material, and the upstream coil 34 and the downstream coil 35 are omitted, it is possible to obtain the effect that the heat load and the power supply cost can be suppressed. Moreover, since the leakage magnetic field from the peeler magnetic field region 44 and the regenerator magnetic field region 45 is suppressed particularly by the magnetic field correction shim 37, the orbit of the beam is less likely to be disturbed before reaching the extraction energy, and the beam can be accelerated more stably.

Further, if the upstream coil 34 and the downstream coil 35 are also used in addition to the magnetic material for forming the peeler magnetic field region 44 and the regenerator magnetic field region 45, the magnetic field strength adjustment of the first and second disturbance magnetic field regions aiming at efficient extraction of the beam becomes possible.

Further, as the extraction radiofrequency wave, a radiofrequency wave that increases the betatron oscillation amplitude in the beam orbit plane of the energy to be extracted and in the direction orthogonal to the beam orbit is fed to the radiofrequency kicker 70. The beam extraction current can be controlled by controlling at least one of the voltage amplitude, phase, frequency, and feeding time of the extraction radiofrequency wave.

Furthermore, the arithmetic unit 91 for controlling the feeding timing of the acceleration radiofrequency wave by the acceleration radiofrequency acceleration system and the feeding timing of the extraction radiofrequency wave by the radiofrequency kicker 70 is further provided. The arithmetic unit 91, after accelerating the beam to a desired energy, starts cutoff of the acceleration radiofrequency wave, and then starts feeding of the extraction radiofrequency wave, supplies the excitation current to the septum coil of the septum magnet 43, 43A, 43B before the extraction of the beam is started, and after feeding of the extraction radiofrequency wave is ended, cuts off the excitation current of the septum coil.

For example, by stopping feeding of the extraction radiofrequency wave to the radiofrequency kicker 70 at an arbitrary timing, the beam does not reach the peeler magnetic field region 44 and the regenerator magnetic field region 45, and the beam extraction from the circular accelerator 39 can be interrupted.

Further, by restarting the feeding to the radiofrequency kicker 70, if the orbital charge remains, it is also possible to restart the beam extraction without injecting, capturing, or accelerating the beam again.

Further, by appropriately controlling the voltage amplitude of the extraction radiofrequency wave fed to the radiofrequency kicker 70, it is possible to absorb the factors that affect the stability of the beam and extract a stable beam with small time variation of the beam current.

That is, since the extraction beam charge for each acceleration cycle can be controlled with high accuracy by the extraction radiofrequency wave, it is possible to perform dose control suitable for scanning. In addition, since the orbital charge can be fully taken out and the scatterer is not required for energy change, the dose rate increases, and the irradiation time can be shortened to improve the patient throughput of the particle therapy system.

Further, the arithmetic unit 91 can shorten the time until the beam is extracted by weakening the electric field of the extraction radiofrequency wave after the feeding of the extraction radiofrequency wave is started and before the beam reaches the disturbance magnetic field region.

### REFERENCE SIGNS LIST

1 last one turn orbit
2 main magnetic field
3 inner peripheral side shim
4 outer peripheral side shim
5 inner peripheral side septum coil conductor
6 outer peripheral side septum coil conductor
7 coil conductor connecting portion
8 coil lead-out portion
10 bipolar power supply
11 acceleration gap
12 dee electrode
13 dummy dee electrode
14 inner conductor
15 outer conductor
20 input coupler
21 pickup loop
22 cathode resistance
23 plate DC power supply
24A, 24B triode
25 acceleration radiofrequency power supply
26 plate DC power supply
30 rotating condenser
31 motor
32 fixed electrode
33 rotating electrode
34 upstream coil (disturbance magnetic field region forming unit, disturbance magnetic field forming coil)
35 downstream coil (disturbance magnetic field region forming unit, disturbance magnetic field forming coil)
36 magnetic field gradient shim (disturbance magnetic field region forming unit, magnetic pole piece)
37 magnetic field correction shim (disturbance magnetic field region forming unit, magnetic pole piece)
38 main magnetic pole
39 circular accelerator
40 main magnet
41 return yoke
42 main coil
43, 43A, 43B septum magnet
44 peeler magnetic field region (first disturbance magnetic field region)
45 regenerator magnetic field region (second disturbance magnetic field region)
47 high energy beam transport
70 radiofrequency kicker (extraction radiofrequency acceleration system)
71 ground electrode
71A, 72A beam passage opening
72 high-voltage electrode
73 projection
80 maximum extraction energy orbit
81 minimum extraction energy orbit
82 beam extraction path entrance
86 radiofrequency kicker power supply
87 upstream coil power supply
88 downstream coil power supply
89 grid bias power supply
90 angle detection mechanism
91 arithmetic unit
92 original oscillator
93 switch
94 preamplifier
95 beam monitor
100 upper shim
101 lower shim
190 rotating gantry
191 control system
192 irradiation system
200 patient
201 treatment table
300 particle therapy system

1. A closed-orbit accelerator that accelerates a beam by a main magnetic field and a frequency-modulated radiofrequency electric field, the accelerator comprising:
an acceleration radiofrequency acceleration system capable of frequency modulation and feeding an acceleration radiofrequency wave for accelerating the beam;
an extraction radiofrequency acceleration system that feeds an extraction radiofrequency wave different in frequency from the acceleration radiofrequency wave for extracting a beam;
a disturbance magnetic field region forming unit that forms a disturbance magnetic field region including a high-order magnetic field that includes a magnetic field component having a number of poles of two poles or more and that includes at least a quadrupole magnetic field component; and
a magnetic shim and a septum magnet having a septum coil.

2. The accelerator according to claim 1, wherein
the septum magnet further has a bipolar power supply for supplying bipolar current to the septum coil.

3. The accelerator according to claim 2, wherein
the shim is composed of an outer peripheral side shim disposed on an outer peripheral side of a beam closed orbit with respect to the septum coil.

4. The accelerator according to claim 2, wherein
the shim is composed of an inner peripheral side shim disposed on an inner peripheral side of the beam closed orbit with respect to the septum coil and an outer peripheral side shim disposed on an outer peripheral side of the beam closed orbit with respect to the septum coil.

5. The accelerator according to claim 4, wherein
the inner peripheral side shim has a wedge shape that does not interfere with a beam closed orbit of the highest energy among the beams.

6. The accelerator according to claim 4, wherein
the inner peripheral side shim and the outer peripheral side shim are independently arranged without being connected to each other.

7. The accelerator according to claim 4, further comprising
an upper shim disposed vertically above the beam closed orbit and a lower shim disposed vertically below the beam closed orbit, wherein
at least one of the inner peripheral side shim and the outer peripheral side shim is connected to the upper shim and the lower shim.

8. The accelerator according to claim 1, wherein
the shim is a laminated steel plate core, and
the septum coil is composed of a coil with ten turns or less.

9. The accelerator according to claim 1, wherein
a magnetic field distribution formed by the main magnetic field is formed so that an injection point of the accelerating beam shifts from the center of the accelerator to an extraction side, and
the disturbance magnetic field region forming unit is arranged at least at two or more places in a location distant from the beam closed orbit under acceleration to the outer peripheral side.

10. The accelerator according to claim 9, wherein
the disturbance magnetic field region forming unit is arranged at two places,
one disturbance magnetic field region forming unit is used as a first disturbance magnetic field region having a magnetic field gradient in which the main magnetic field is weakened toward the radial outer peripheral side, and
the other disturbance magnetic field region forming unit is used as a second disturbance magnetic field region having a magnetic field gradient in which the main magnetic field is strengthened toward the radial outer peripheral side.

11. The accelerator according to claim 10, wherein
the disturbance magnetic field region forming unit includes any one of a magnetic pole piece made of a magnetic material, a disturbance magnetic field forming coil, and both of the magnetic pole piece and the disturbance magnetic field forming coil.

12. The accelerator according to claim 1, wherein
the extraction radiofrequency acceleration system
feeds, as the acceleration radiofrequency wave, a radiofrequency wave that increases a betatron oscillation amplitude in a beam orbit plane of energy to be extracted, and in a direction orthogonal to an orbit of the beam, and
controls at least one of the electric field amplitude, phase, frequency, and feeding time of the extraction radiofrequency wave to control a charge amount of an extracted beam pulse and a time structure of the beam pulse.

13. The accelerator according to claim 1, further comprising
an arithmetic unit for controlling a feeding timing of the acceleration radiofrequency wave by the acceleration radiofrequency acceleration system and a feeding timing of the extraction radiofrequency wave by the extraction radiofrequency acceleration system, wherein the arithmetic unit
after accelerating the beam to a desired energy, starts cutoff of the acceleration radiofrequency wave, and then starts feeding of the extraction radiofrequency wave, and
supplies an excitation current to the septum coil of the septum magnet before the extraction of the beam is started, and cuts off the excitation current of the septum coil after ending the feeding of the extraction radiofrequency wave.

14. The accelerator according to claim 13, wherein
the arithmetic unit further weakens the electric field of the extraction radiofrequency wave after start of the feeding of the extraction radiofrequency wave and before the beam reaches the disturbance magnetic field region.

15. A particle therapy system comprising:
an accelerator according to claim 1; and
an irradiation system that irradiates a beam of specific energy extracted from the accelerator.
